(19) United States
(12) Patent Application Publication
(10) Pub. No.: US 2026/0021280 A1
(43) Pub. Date: Jan. 22, 2026

(54) BALLOON CATHETER AND METHOD OF USE FOR DELIVERING A STENT TO THE VENOUS SINUSES

(71) Applicant: Serenity Medical, Inc., Deephaven, MN (US)

(72) Inventor: Jeffrey P. Callister, Deephaven, MN (US)

(21) Appl. No.: 19/276,638

(22) Filed: Jul. 22, 2025

Related U.S. Application Data
(60) Provisional application No. 63/673,861, filed on Jul. 22, 2024.

(51) Int. Cl. A61M 25/10; A61M 25/00

(52) U.S. Cl. CPC A61M 25/10; A61M 25/0045; A61M 2025/1093; A61M 2205/0222; A61M 2205/0238; A61M 2205/32

(57) ABSTRACT

A balloon catheter includes a hub, a shaft, a balloon, and a tip. A shaft proximal end is coupled to a hub distal end. At least a portion of the shaft includes a balloon inflation lumen and a first portion of a guidewire lumen. A balloon proximal end is coupled to a shaft distal end. A second portion of the guidewire lumen extends at least partially through the balloon. The tip includes a medical-grade polymer. A third portion of the guidewire lumen extends through the tip from a tip proximal end to a tip distal end. A balloon distal end is bonded to the tip at a balloon-tip junction towards the tip proximal end. A tip length from the tip distal end to the balloon distal end is seven (7) to fifty (50) millimeters in a straightened state of the tip.

### CROSS-REFERENCE TO RELATED APPLICATIONS/INCORPORATION BY REFERENCE

The present application claims priority under 35 U.S.C. § 119 (e) to provisional application Ser. No. 63/673,861 filed on Jul. 22, 2024, entitled “BALLOON CATHETER AND METHOD OF USE FOR DELIVERING A STENT TO THE VENOUS SINUSES.” The above referenced provisional application is hereby incorporated herein by reference in its entirety.

U.S. Pat. No. 10,940,030 by Callister, which issued to Serenity Medical, Inc. on Mar. 9, 2021, filed Mar. 10, 2017, and titled “METHOD AND SYSTEM FOR DELIVERING A SELF-EXPANDING STENT TO THE VENOUS SINUSES,” is incorporated by reference herein in its entirety.

U.S. Pat. No. 11,717,430 by Callister, which issued to Serenity Medical, Inc. on Aug. 8, 2023, filed Mar. 5, 2021, and titled “METHOD AND SYSTEM FOR DELIVERING A SELF-EXPANDING STENT TO THE VENOUS SINUSES,” is incorporated by reference herein in its entirety.

U.S. patent application Ser. No. 18/230,785, filed Aug. 7, 2023, and titled “METHOD AND SYSTEM FOR DELIVERING A SELF-EXPANDING STENT TO THE VENOUS SINUSES,” is incorporated by reference herein in its entirety.

### FIELD

Certain embodiments relate to systems and methods for inserting/navigating a stent dilatation balloon catheter into various venous sinuses. More specifically, certain embodiments include crossing the sigmoid junction for placement and dilatation of a stent within the transverse sinus, sigmoid sinus, and other areas such as the sagittal sinus. Additionally, various embodiments describe accessing and crossing through stents placed in these locations and dilatation thereof.

### BACKGROUND

When blood exiting the brain is slowed by a restriction in the venous sinuses, it causes an increase to the distal blood pressure, which may translate to an increase in the brain fluid pressure. Patients experiencing Increased Intracranial Pressure (ICP), where the Cerebral Spinal Fluid (CSF) pressure in the cranium has increased, may suffer from headaches, loss of vision, and/or tinnitus, among other things. Previous methods for treating a collapse of and/or a stenosis in the sigmoid and/or transverse sinus has been drugs and/or using a shunt to relieve the CSF fluid pressure. The use of drugs or a shunt is not ideal, however, because both are temporary solutions that each carry associated risks.

More, recently, venous sinus occlusions are being stented to reduce or eliminate idiopathic intracranial hypertension (IIH) and/or tinnitus. The procedure involves placing a stent in the venous sinus system of patients to ameliorate a collapse of and/or a stenosis in the sigmoid and/or transverse sinus and to restore improved blood flow out of the brain. Note that the venous sinus structure does not resemble any vein or arteries of other parts of the body. Instead, the venous sinus is a void created where the dura joins and forms a cavity (i.e., sinus) primarily along the inside of the skull. The dura has no smooth muscle cell lining and is inelastic when compared to veins and arteries.

FIG. 1 illustrates an exemplary venous sinus system having an identified stent zone. The venous sinus system comprises venous channels found between the periosteal and meningeal layers of dura mater in the brain. The venous sinus system receives blood from internal and external veins of the brain, receives CSF from the subarachnoid space via arachnoid granulations, and mainly empties into the internal jugular vein. As illustrated in FIG. 1, the venous sinus system includes the transverse sinus, sigmoid sinus, and the sigmoid junction. The sigmoid sinus integrates into the jugular vein at the sigmoid junction. FIG. 1 also identifies an exemplary stent zone for placing a stent to treat a collapse of and/or a stenosis in the sigmoid and/or transverse sinus.

Prior to stenting, a balloon catheter may be used to open a blockage and/or a restriction in the passageway in the sinus to facilitate easier placement and delivery of a stent; and/or to block the sigmoid sinus to allow the physician to determine if the patient has tinnitus caused by flow irregularities in the sinus pathways. Additionally, or alternatively, a balloon catheter may be used to further open the stent after stenting. Currently, the procedure of crossing the sigmoid junction with off-the-shelf stent catheters and balloon dilation catheters can be challenging. Specifically, navigating through the sigmoid junction with a guidewire, then positioning a suitably sized guide catheter (GC) across the wire, through the sigmoid junction, into the sigmoid sinus, and sometimes advancing the GC to the distal end of the transverse sinus is a difficult task. The GC tip is difficult to maneuver across the sigmoid junction and a repetitive process involving pushing, torquing, pushing, torquing, etc. until finally getting the GC tip past the junction may take a substantial amount of time to complete. For example, the process can take anywhere from a few minutes to over 45 minutes depending on the severity of the bends/tortuosity encountered in the sigmoid junction, as shown in FIG. 1. This present procedure sometimes involves crossing the junction with a suitable sized guide wire, then advancing a microcatheter over the wire for reinforcement, then advancing to larger outer diameter (OD) GC until the suitable sized guide catheter is positioned. Once the suitable sized guide catheter and 0.014″ guide wire is in place across the sigmoid junction, the GC serves as a conduit for advancing the stent catheter and/or other guide catheters, balloon catheters, and/or micro catheters through this difficult to cross area.

The sigmoid junction comes in a variety of different tortuosity. In some cases, this tortuosity can comprise two turns, with one turn out of plane, and with radiuses up to 140 degrees in bend, which is a difficult passage to cross with current off-the-shelf stents and balloon catheters.

The unique design of the stent catheter and stent described in U.S. patent application Ser. No. 18/230,785 and U.S. Pat. Nos. 10,940,030 and 11,717,430, which are incorporated by reference herein in their entirety, eliminates the tedious pre-procedures of crossing the sigmoid junction with an array of catheters to get the appropriately sized GC across the sigmoid junction to serve as a conduit. The procedure described in U.S. patent application Ser. No. 18/230,785 and U.S. Pat. Nos. 10,940,030 and 11,717,430 involves advancing the suitable sized guide catheter up to the distal end of the jugular (a straight shot); advancing a 0.014″ guide wire through the guide and through the sigmoid junction (note: getting a 0.014″ across can sometimes be difficult due to the individual anatomy); and, after crossing the sigmoid junction, advancing the guidewire and anchoring the distal wire tip in either the sagittal or the opposing transverse sinus. The innovative tip/catheter/stent and Rapid Exchange (RX) design described in U.S. patent application Ser. No. 18/230,785 and U.S. Pat. Nos. 10,940,030 and 11,717,430 enables it to effortlessly track along the 0.014″ guide wire, navigate the difficult sigmoid junction, and be positioned accurately at the desired location with no GC support in the sigmoid junction.

In the case where a stent might require a balloon dilatation to further open the stent diameter, the sigmoid junction may have to be traversed, again, with a GC to facilitate getting a ballon catheter across the junction, which may add unnecessary procedure time to the patient. This additional procedure time is eliminated when using the catheter described in U.S. patent application Ser. No. 18/230,785 and U.S. Pat. Nos. 10,940,030 and 11,717,430.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY

Systems and methods for inserting/navigating a stent dilatation balloon catheter into various venous sinuses are provided, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 illustrates an exemplary venous sinus system having an identified stent zone, in accordance with various embodiments.

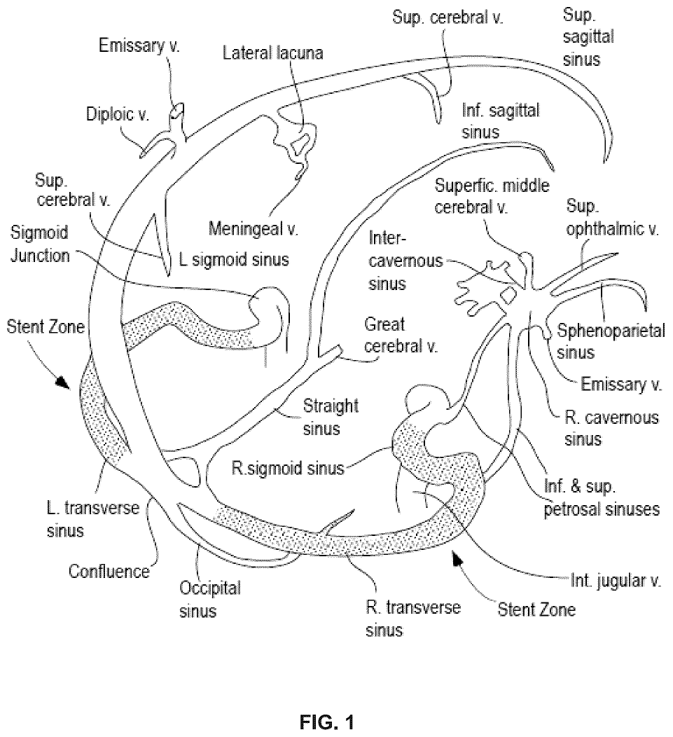

FIG. 2A illustrates a perspective view of an exemplary balloon catheter having a lengthy tip, in accordance with various embodiments.

FIG. 2B illustrates a cross-sectional view of an exemplary Rapid Exchange (RX) joint shown in FIG. 2A, in accordance with various embodiments.

FIG. 3 illustrates a cross-sectional view of an exemplary lengthy tip of a balloon catheter, in accordance with various embodiments.

FIG. 4 illustrates a cross-sectional view of an exemplary lengthy tip of a balloon catheter having increasing outer diameters from a distal end of the tip to a balloon-tip junction, in accordance with various embodiments.

FIG. 5 illustrates a cross-sectional view of an exemplary lengthy tip of a balloon catheter having a J-bend of less than 90 degrees, in accordance with various embodiments.

FIG. 6 illustrates a cross-sectional view of an exemplary lengthy tip of a balloon catheter having a J-bend of greater than 90 degrees, the lengthy tip of the balloon catheter in a collapsed state and navigated to a constriction in a stented portion of the venous sinuses, in accordance with various embodiments.

### DETAILED DESCRIPTION

Certain embodiments may provide systems and methods for inserting/navigating a stent dilatation balloon catheter into various venous sinuses. Various embodiments provide innovative design features to balloon catheter tip elements, such as lengthy tips, progressive use of durometers to increase stiffness going proximally, variable diameters to change flexibility, lubricious coatings on outer diameters, lubricious liners on the inside diameter, and unique distal tip configurations to enhance navigation through tortuous stents. These enhancements improve the balloon catheter's ability to advance over a guide wire and through a tortuous sigmoid junction using a 0.014″ guidewire track-line. This approach can also pertain and use up to a 0.018″ guidewire. This approach replaces the intensive procedure of placing a properly sized guide catheter across the sigmoid junction to facilitate a conduit to cross the sigmoid junction. Accordingly, a balloon catheter may easily navigate the sigmoid junction over a 0.014″ guidewire or any suitably sized guidewire, allowing for the pre-dilation of the desired sinus area, including dilation and occlusion of the sigmoid sinus for tinnitus evaluation, and/or the dilation of a stent to the appropriate size without further catheterization.

The foregoing summary, as well as the following detailed description of certain embodiments will be better understood when read in conjunction with the appended drawings. It should be understood that the various embodiments are not limited to the arrangements and instrumentality shown in the drawings. It should also be understood that the embodiments may be combined, or that other embodiments may be utilized and that structural changes may be made without departing from the scope of the various embodiments. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property. As referred to herein, the terms “proximal” and “distal” are in relation to a catheter hub held by a doctor. For example, the distal end is farther from the catheter hub and proximal end is closer to the catheter hub.

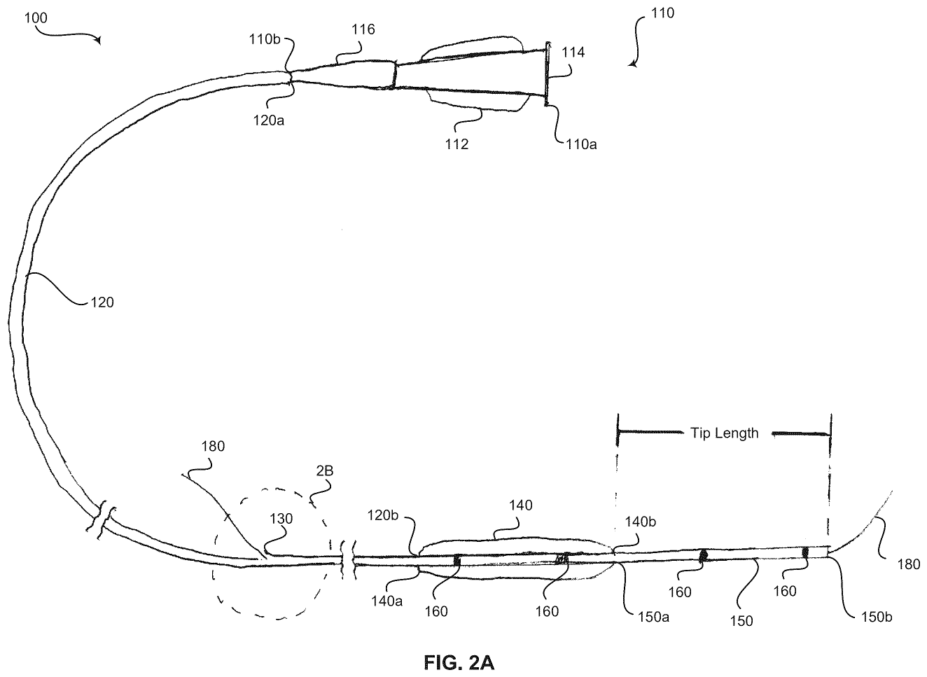

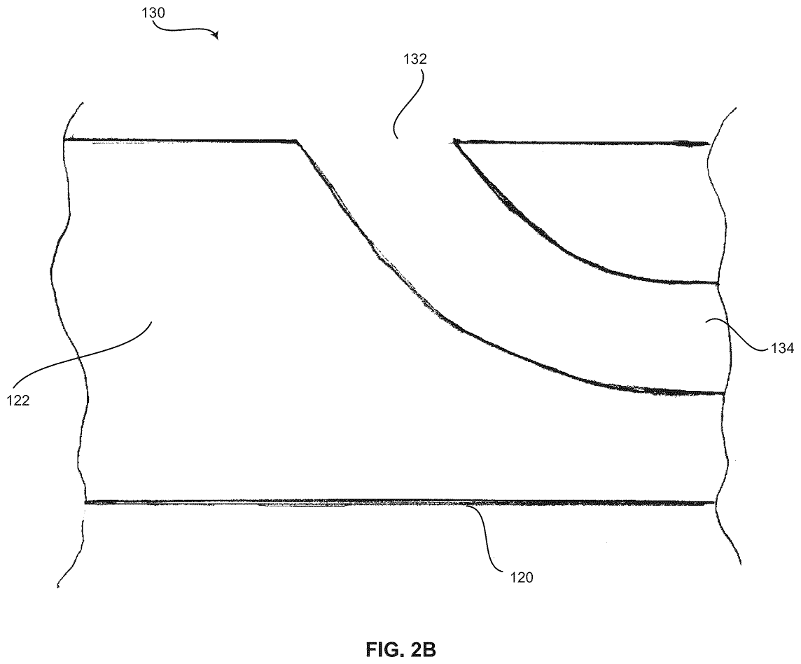

FIG. 2A illustrates a perspective view of an exemplary balloon catheter 100 having a lengthy tip 150, in accordance with various embodiments. Referring to FIG. 2A, a Rapid Exchange (RX) balloon catheter is shown. However, the present application is not limited to RX balloon catheters. Instead, the balloon catheter may be an over the wire (OTW) balloon catheter, monorail balloon catheter, and/or any suitable balloon catheter. The balloon catheter 100 may comprise a hub 110, a shaft 120, a balloon 140, an extended tip 150, and a guidewire 180, among other things.

The hub 110 (also referred to as a handle) may comprise a hub proximal end 110a and a hub distal end 110b. The hub 110 of an RX balloon catheter 100 may comprise a Luer wing 112 having a balloon inflation port 114 at the proximal end 110a of the hub 110. The balloon inflation port 114 provides access to a balloon inflation lumen that carries balloon inflation fluid via the hub 110 and shaft 120 between a balloon fluid source (not shown) and the balloon 140 to inflate and deflate the balloon 140. The Luer wing 112 of the hub 110 may be coupled to a proximal end of the shaft 120a at the distal end 110b of the hub 110 by a strain relief 116, for example. The strain relief 116 protects the connection between the shaft 120 and the hub 110 from heavy loads caused by an operator pulling, pushing, twisting, or bending the balloon catheter 100. In embodiments comprising an over the wire (OTW) balloon catheter, the hub 110 of the OTW balloon catheter may comprise a guidewire lumen port (not shown).

The shaft 120 may be a tubular structure made of nitinol, stainless steel tubing, medical-grade polymers, such as polyurethane, polyether block amide, and/or low-density polyethylene (LDPE), and/or any suitable material. The shaft 120 comprises a proximal end 120a coupled to a distal end 110b of the hub and a distal end 120b coupled to a proximal end 140a of the balloon 140. In various embodiments, the shaft 120 may comprise multiple segments made of different materials and/or having different outside diameters, for example, to increase flexibility from the shaft proximal end 120a along a longitudinal axis to the shaft distal end 120b. In an OTW balloon catheter (not shown), the shaft 120 comprises concentric lumens extending from the hub 110 to the balloon 140. The concentric lumens include an inner guidewire lumen and an outer balloon inflation lumen. The outer balloon inflation lumen terminates at the distal end 120b of the shaft 120. The inner guidewire lumen extends from a guidewire lumen port at the hub 110, through the shaft 120, out of the distal end 120b of the shaft 120, through the balloon 140, and through the tip 150 to a distal end 150b of the tip 150.

In an RX balloon catheter 100 as shown in FIG. 2A, the shaft comprises a balloon inflation lumen that extends from the hub 110 to the balloon 140. The shaft 120 comprises an RX joint 130 between the hub 110 and the balloon 140. The RX joint 130 includes an RX port that introduces a guidewire lumen, which may be arranged within the balloon inflation lumen. Accordingly, the shaft 120 may comprise a single lumen (i.e., the balloon inflation lumen) between the hub 110 and the RX joint 130, and concentric lumens (i.e., a guidewire lumen arranged within the balloon inflation lumen) from the RX joint 130 to the balloon 140. A first portion of the guidewire lumen may extend from the RX port and out of the distal end 120b of the shaft 120. A second portion of the guidewire lumen may extend through the balloon 140. A third portion of the guidewire lumen may extend through the tip 150 to a distal end 150b of the tip 150. The guidewire lumen is configured to allow a guidewire 180 to pass through the guidewire lumen such that the balloon catheter 100 may glide over the guidewire 180 during navigation of the catheter to the venous sinuses or other body lumen. The guidewire 180 may be a 0.014″ guidewire, a 0.018″ guidewire, or any suitably sized guidewire. In various embodiments, an inside diameter of the guidewire lumen may have a polytetrafluoroethylene (PTFE) liner to reduce friction of movement over the guidewire 180 and/or incorporate a highly PTFE loaded biocompatible polymer, such as a polyimide thin-walled tube. In various embodiments, at least a part of the guidewire lumen is formed by a polymer coated braided shaft.

FIG. 2B illustrates a cross-sectional view of an exemplary Rapid Exchange (RX) joint 130 shown in FIG. 2A, in accordance with various embodiments. Referring to FIG. 2B, the shaft 120 comprises a balloon inflation lumen 122 and the RX joint 130. The RX joint 130 comprises an RX port 132 providing access to a guidewire lumen 134 that extends from the Rx port 132 through the distal end of a tip at the distal end of a balloon catheter. A guidewire (not shown) may be inserted through the RX port 132 and guidewire lumen 134 such that the balloon catheter can slide over the guidewire during navigation of the balloon catheter to the venous sinuses or other body lumen. In an exemplary embodiment, the balloon inflation lumen 122 may be configured to carry fluid between a balloon inflation port 114 of the hub 110 and the balloon 140 to inflate and deflate the balloon 140. The guidewire lumen 134 may be arranged within the balloon inflation lumen 122. For example, the guidewire lumen 134 and balloon inflation lumen 122 may be arranged concentrically. In various embodiments, an inside diameter of the guidewire lumen 134 may have a polytetrafluoroethylene (PTFE) liner to reduce friction of movement over a guidewire and/or incorporate a highly PTFE loaded biocompatible polymer, such as a polyimide thin-walled tube. In various embodiments, at least a part of the guidewire lumen is formed by a polymer coated braided shaft.

Referring again to FIG. 2, the balloon 140 is a thin, flexible material, such as polyurethane, polyether block amide (e.g., PEBAX), polyethylene (PE), nylon, polyethylene terephthalate (PET), and/or any suitable material. The balloon 140 comprises a proximal end 140a coupled to the distal end 120b of the shaft 120, and a distal end 140b coupled toward a proximal end 150a of the tip 150. The balloon is configured to expand and contract by receiving fluid from, or removing fluid to, a fluid source coupled to the balloon inflation port 114 of the hub 110. The guidewire lumen 134 extends through the balloon 140. The balloon design incorporates both a monorail balloon design and preferably a Rapid Exchange (RX) design. In an exemplary embodiment, the balloon sizes are 4-8 mm in diameter (preferably 4-6 mm), and are 2-6 cm long (preferably 3 cm). The balloon crossing profiles may be compatible with 5 Fr guiding catheter, 6 Fr guiding catheter, or larger sizes.

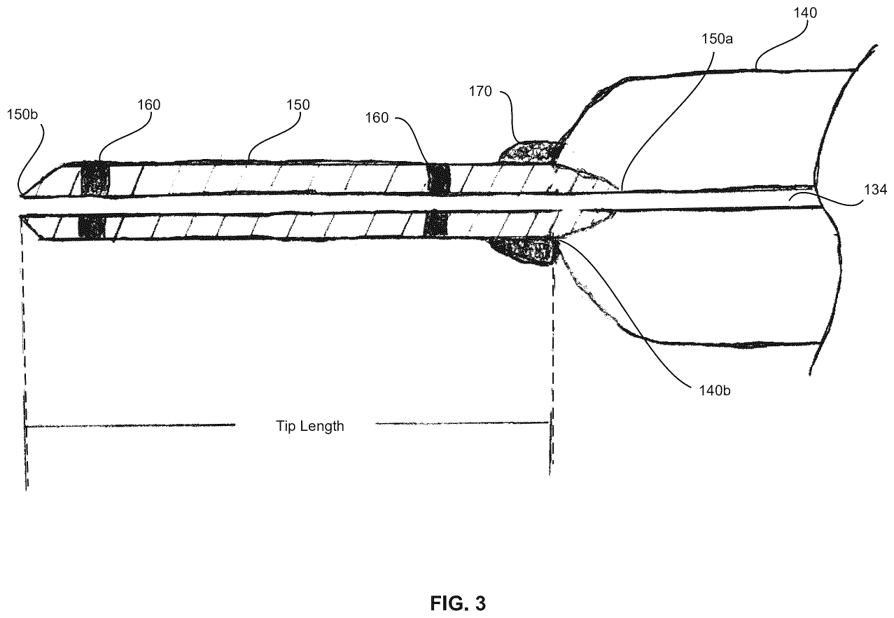

The tip 150 comprises various medical-grade polymers formed over a third portion of the guidewire lumen 134. The tip 150 comprises a proximal end 150a and a distal end 150b. The distal end 140b of the balloon 140 is bonded toward the proximal end 150a of the tip. In various embodiments, a portion of the material of the tip 150 may extend within the balloon 140 as shown below in FIGS. 3-5, for example. In various embodiments, the tip length may be in a range from 7 millimeters (mm) to 50 mm. In other embodiments, the tip length may be in a range from 12 mm to 45 mm. In a preferred embodiment, the tip length may be in a range from 25 mm to 35 mm. For purpose of the present application, the tip length is defined as the length of the tip 150 in a straightened state from the distal end 150b of the tip 150 to the distal end 140b of the balloon 140 bonded to the tip 150. Accordingly, the tip length does not include the most proximal portion of the material of the tip 150 that may extend slightly within the balloon 140. The lengthy, flexible tip 150 serves as a mechanism that easily tracks the guidewire 180. Once the lengthy tip 150 tracking the guidewire 180 is through and past the sigmoid junction of the venous sinuses, the ballon portion 150 of the catheter follows (i.e. by being pushed forward). Here it is like a train car, once the engine is past a difficult section of track the rest of the train cars follow.

In certain embodiments, an outside surface of tip 150 may be coated with slip agents, such as hydrophilic coatings or various other medical lubricants, to facilitate easier advancement through both the guide catheters and venous tracks. The inside diameter of the tip 150 (i.e., the guidewire lumen 134) may have a polytetrafluoroethylene (PTFE) liner to further reduce friction of movement over the guidewire and/or incorporate a highly PTFE loaded biocompatible polymer, such as a polyimide thin-walled tube.

In various embodiments, the guidewire lumen 134 and/or tip 150 may include one or more markers 160, such as radio-opaque markers 160, to enhance visualization of the location of the balloon catheter 100. For example, an operator of the balloon catheter 100 may monitor the navigation of the balloon catheter 100 via medical image data, such as fluoroscopic images, ultrasound images, or images of any suitable medical imaging modality. The marker(s) 160 may be readily identifiable in the image data to assist the operator in accurately positioning the balloon catheter 100 at the appropriate location in the venous sinuses.

FIG. 3 illustrates a cross-sectional view of an exemplary lengthy tip 150 of a balloon catheter, in accordance with various embodiments. Referring to FIG. 3, the lengthy tip 150 comprises a various medical-grade polymers formed over a third portion of the guidewire lumen 134. The tip 150 comprises a proximal end 150a and a distal end 150b. The distal end 140b of the balloon 140 is bonded by heat welding, laser welding, and/or a with a bonding adhesive 170 toward the proximal end 150a of the tip with a portion of the material of the tip 150 extending within the balloon 140. The various medical-grade polymers may include polyurethane, polyether block amide (e.g., PEBAX), and/or low-density polyethylene (LDPE). The polymer durometer may be a single low mono durometer or preferably constructed using a variation of softer to stiffer polymers up to the balloon tip junction 140b, 150, 170. Preferably, the tip durometer ranges from 80A to 45D, with an overall range encompassing from 70A to 55D. In various embodiments, the tip length (i.e., in a straightened state from the distal end 150b of the tip 150 to the distal end 140b of the balloon 140 bonded to the tip 150) may be in a range from 7 mm to 50 mm. In other embodiments, the tip length may be in a range from 12 mm to 45 mm. In a preferred embodiment, the tip length may be in a range from 25 mm to 35 mm. In an exemplary embodiment, the tip 150 may include one or more radio-opaque markers 160 configured to enhance visualization of the location of the balloon catheter 100 in medical imaging data.

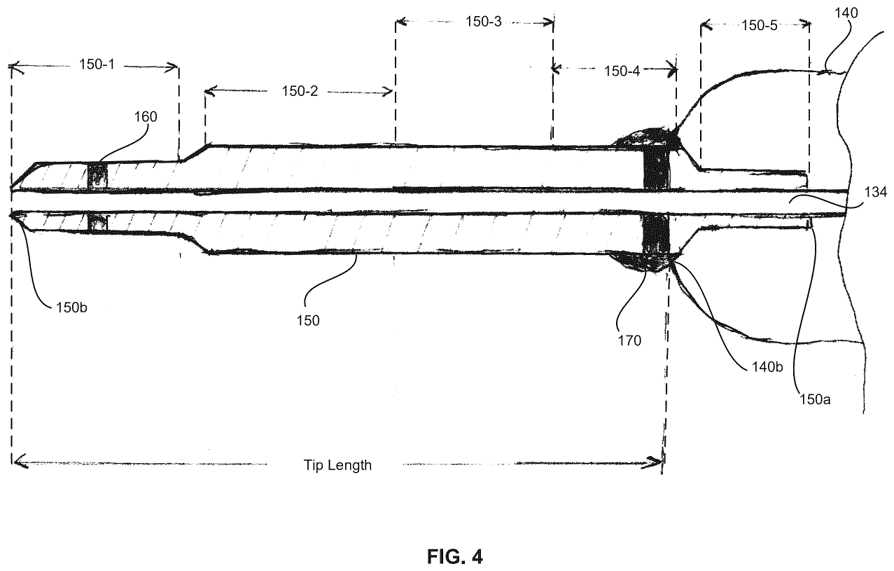

FIG. 4 illustrates a cross-sectional view of an exemplary lengthy tip 150 of a balloon catheter having increasing outer diameters from a distal end 150b of the tip to a balloon-tip junction 140b, 150, 170, in accordance with various embodiments. Referring to FIG. 4, the lengthy tip 150 comprises a various medical-grade polymers formed over a third portion of the guidewire lumen 134. The tip 150 comprises a proximal end 150a and a distal end 150b. The distal end 140b of the balloon 140 is bonded 170 toward the proximal end 150a of the tip with a portion of the material of the tip 150 extending within the balloon 140. The various medical-grade polymers may include polyurethane, polyether block amide (e.g., PEBAX), and/or low-density polyethylene (LDPE). The tip 150 may include a plurality of segments 150-1, 150-2, 150-3, 150-4, 150-5 each having an outer diameter and a durometer. In an exemplary embodiment, each of the plurality of segments 150-1, 150-2, 150-3, 150-4, 150-5 may be at least 2 mm long. Still referring to FIG. 4, the exemplary progressive lengthy tip 150 may be constructed with a lower durometer in the forward tip segment 150-1 with a smaller outer diameter (OD) that progressively increases to a higher durometer and/or OD at a segment 150-4 proximal to the balloon tip junction 140b, 150, 170, which demonstrates a progressive durometer tip design. The lengths and diameters of the differing durometer segments 150-1, 150-2, 150-3, 150-4, 150-5 may be varied to achieve an integrally progressive transition from a flexible to a stiffer tip 150—distal 150b to proximal 150a. Adjusting the lengths of the durometer segments 150-1, 150-2, 150-3, 150-4, 150-5 allows for a tip 150 that progressively changes from flexible to stiff. In addition, the progressive increase in flexibility from the distal end 150b of the tip 150 to the proximal end 150a of the tip 150 provides enhanced trackability and easier pushability over tight bends in the guidewire. For example, a first durometer of segment 150-1 may be between 70A and 40D, a second durometer of segments 150-2, 150-3, and 150-4 may be between 40D and 45D, and a third durometer of segment 150-5 may be between 45D and 55D. As another example, a first durometer of segment 150-1 may be between 70A and 35D, a second durometer of segment 150-2 may be greater than the first durometer and between 30D and 38D, a third durometer of segment 150-3 may be greater than the second durometer and between 35D and 40D, a fourth durometer of segment 150-4 may be between 40D and 45D, and fifth durometer of segment 150-5 may be between 45D and 63D. Although not shown in FIG. 4, various embodiments provide a smooth transition from larger diameter at the balloon tip junction 140b, 150, 170 to a smaller diameter at the distal end 150b of the tip 150, such as a truncated conical shape. In an exemplary embodiment, the tip 150 may comprise one and/or multiple radiopaque markers (RM) 160 embedded in the tip 150 to show the tip location under x-ray fluoroscopy and serve as length measurements.

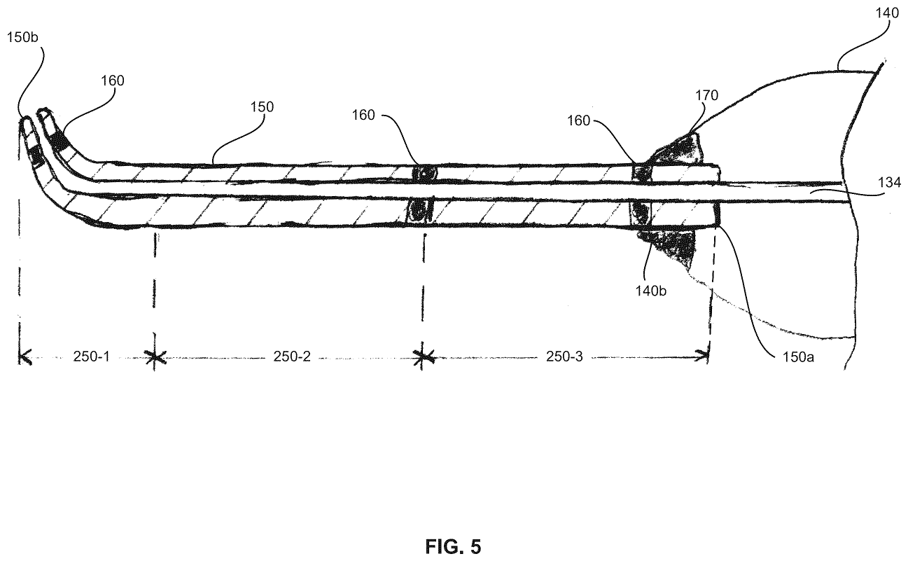

FIG. 5 illustrates a cross-sectional view of an exemplary lengthy tip 150 of a balloon catheter having a J-bend of less than 90 degrees, in accordance with various embodiments. Referring to FIG. 5, the lengthy, J-bend tip 150 comprises a various medical-grade polymers formed over a third portion of the guidewire lumen 134. The tip 150 comprises a proximal end 150a and a distal end 150b. The distal end 140b of the balloon 140 is bonded 170 toward the proximal end 150a of the tip with a portion of the material of the tip 150 extending within the balloon 140. Examples of the various medical-grade polymers may include polyurethane, polyether block amide (e.g., PEBAX), and/or low-density polyethylene (LDPE). The tip 150 may include a plurality of segments 250-1, 250-2, 250-3, each having a durometer. In an exemplary embodiment, each of the plurality of segments 250-1, 250-2, 250-3 may be at least 2 mm long. The exemplary lengthy, J-bend tip 150 may be constructed with a lower durometer in the forward tip segment 250-1 with a smaller durometer that progressively increases to a higher durometer at a segment 250-3 proximal to the balloon tip junction 140b, 150, 170, which demonstrates a progressive durometer tip design. For example, a first durometer of segment 250-1 may be between 70A and 38D, a second durometer of segment 250-2 is greater than the first durometer and may be between 30 and 45D, and third durometer of segment 250-3 is greater than the second durometer and may be between 40D and 55D. Still referring to FIG. 5, a distal portion 250-1 of the lengthy tip can have a slight “J” bend in it, which facilitates navigating through a poorly expanded stent. The J-bend is shown as being under 90 degrees; however, the degree of curvature can range from 0 degrees (i.e., straight) up to 180 degrees. For example, a balloon 140 led by a straight tip may lodge up against a stent strut while traversing the guidewire through a tortuous part of the stented sinus anatomy, thereby preventing the balloon 140 from being pushed forward. With a slight ‘J’ shape as shown in FIG. 5, the doctor can slightly torque the balloon catheter shaft 120 at the handle/hub 110 and slightly rotate the tip 150 to disengage the distal end 150b of the tip 150 from the stent wall allowing the ballon catheter 100 to be advanced and then be pushed forward. Note that the J-bend may be in a relaxed state without a guidewire passing through the J-bend portion of the J-bend tip 150. The J-bend may be less pronounced or substantially eliminated when a guidewire passes entirely through the tip 150 depending on the stiffness of the guidewire.

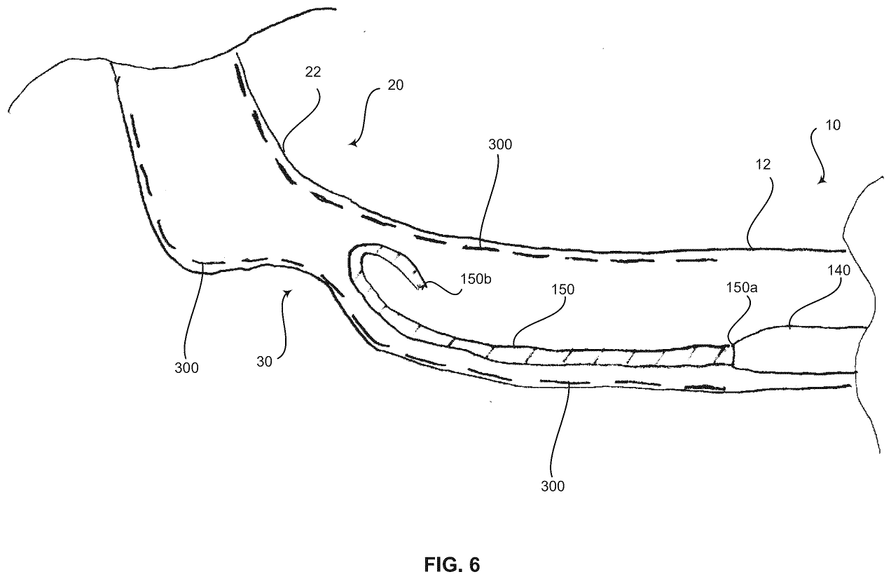

FIG. 6 illustrates a cross-sectional view of an exemplary lengthy tip 150 of a balloon catheter having a J-bend of greater than 90 degrees, the lengthy tip 150 of the balloon catheter in a collapsed state and navigated to a constriction 30 in a stented portion 300 of the venous sinuses 10, 20, in accordance with various embodiments. Referring to FIG. 6, a portion of the venous sinuses having the sigmoid wall 12 of the sigmoid sinus 10, the transverse sinus wall 22 of the transverse sinus 20, and a sinus constriction 30 is shown. Stent struts 300 of a stent provided in the sigmoid 10 and transverse 20 sinuses is also shown. The balloon catheter is being navigated to position the folded balloon 140 within the sinus constriction 30 so that the folded balloon 140 may be inflated to open the constricted sinus 30. In practice, a physician may lose guidewire access across a stent in the sinus, causing the physician to have to recross through an interior of the stent, which may be difficult because the guidewire may (1) get tangled in any of the stent wall openings 300 that are not pressed firmly against the sinus wall 12, 22; (2) pass under a strut wall 300 between stent and sinus wall 12, 22; and/or, (3) not be able to navigate through the inside of the stent at all. By incorporating a J-bend greater than 90 degrees in the tip 150, the guidewire can be pulled inwards from the distal end 150b of the tip 150 such that the guidewire presses against the curved portion of the J-bend. The J-bend of the tip 150 may collapse without the guidewire support and the guidewire may be pushed against the curve in the J-bend within the tip 150 to push the tip 150 through the stent structure 300 and into position for inflation of the balloon 140 to open the sinus constriction 30.

Aspects of the present disclosure provide systems and methods for inserting/navigating a stent dilatation balloon catheter 100 into various venous sinuses. The balloon catheter 100 may comprise a hub 110, a shaft 120, a balloon 140, and a tip 150. The hub 110 may comprise a hub proximal end 110a and a hub distal end 110b. The shaft 120 may comprise a shaft proximal end 120a and a shaft distal end 120b. The shaft proximal end 120a may be coupled to the hub distal end 110b. At least a portion of the shaft 120 may comprise a balloon inflation lumen 122 and a first portion of a guidewire lumen 134. The balloon 140 may comprise a balloon proximal end 140a and a balloon distal end 140b. The balloon proximal end 140a may be coupled to the shaft distal end 120b. A second portion of the guidewire lumen 134 may extend at least partially through the balloon 140. The tip 150 may comprise a tip proximal end 150a and a tip distal end 150b. The tip 150 may comprise a medical-grade polymer. A third portion of the guidewire lumen 134 may extend through the tip 150 from the tip proximal end 150a to the tip distal end 150b. The balloon distal end 140b may be bonded 170 to the tip 150 at a balloon-tip junction towards the tip proximal end 150a. A tip length from the tip distal end 150b to the balloon distal end 140b is seven (7) to fifty (50) millimeters in a straightened state of the tip 150.

In an exemplary embodiment, the tip length is twelve (12) to forty-five (45) millimeters in a straightened state of the tip 150. In a representative embodiment, the tip length is twenty-five (25) to thirty-five (35) millimeters in a straightened state of the tip 150. In various embodiments, the tip 150 comprises a curvature in a relaxed state. In certain embodiments, the curvature is ninety (90) to one hundred and eighty (180) degrees in the relaxed state. In an exemplary embodiment, the tip 150 comprises a first durometer at the tip distal end 150b and a second durometer at the tip proximal end 150a. The first durometer is less than the second durometer. In a representative embodiment, the medical-grade polymer comprises polyurethane, polyether block amide, and/or low-density polyethylene (LDPE). In various embodiments, the tip 150 comprises a first outer diameter at the tip distal end 150b and a second outer diameter at the balloon-tip junction 140b, 150, 170. The first outer diameter is smaller than the second outer diameter. In certain embodiments, an outer diameter of the tip 150 transitions from a first outer diameter at the tip distal end 150b to a second outer diameter, greater than the first outer diameter, at the balloon-tip junction 140b, 150, 170. In an exemplary embodiment, the tip 150 comprises a truncated conical shape with a smallest outer diameter at the tip distal end 150b. In a representative embodiment, the tip 150 comprises a plurality of segments 150-1, 150-2, 150-3, 150-4, 150-5 each having an outer diameter. A first segment 150-1 at the tip distal end 150b has a first diameter. A second segment 150-4 at the ballon-tip junction 140, 150, 170 has a second diameter. A third segment 150-2, 150-3 between the first segment 150-1 and the second segment 150-4 has a third diameter. The second diameter is greater than the third diameter. The third diameter is greater than the first diameter. In various embodiments, each of the plurality of segments 150-1, 150-2, 150-3, 150-4, 150-5 is at least two (2) millimeters long.

In certain embodiments, the tip comprises a plurality of segments 150-1, 150-2, 150-3, 150-4, 150-5, 250-1, 250-2, 250-3 each having a durometer, A first segment 150-1, 250-1 at the tip distal end 150b has a first durometer. A second segment 150-4, 250-3 at the ballon-tip junction 140, 150, 170 has a second durometer. A third segment 150-2, 150-3, 250-2 between the first segment 150-1, 250-1 and the second segment 150-4, 250-3 has a third durometer. The second durometer is greater than the third durometer. The third durometer is greater than the first durometer. In an exemplary embodiment, each of the plurality of segments 150-1, 150-2, 150-3, 150-4, 150-5, 250-1, 250-2, 250-3 is at least two (2) millimeters long. In a representative embodiment, the first durometer is between 70A and 40D, the second durometer is between 45D and 55D, and the third durometer is between 40D and 45D. In various embodiments, at least a portion of the outer surface of the tip 150 is coated with a slip agent. In certain embodiments, the slip agent comprises a hydrophilic coating. In an exemplary embodiment, at least part of the third portion of the guidewire lumen 134 comprises a polytetrafluoroethylene (PTFE) liner. In a representative embodiment, at least part of the third portion of the guidewire lumen 134 comprises a polyimide thin-walled tube. In various embodiments, the tip 150 comprises a plurality of radiopaque markers 160.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a structure that is “configured” to or “operable” to perform a function requires that the structure is more than just capable of performing the function, but is actually made to perform the function, regardless of whether the function is actually performed.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A balloon catheter comprising:
a hub having a hub proximal end and a hub distal end;
a shaft having a shaft proximal end and a shaft distal end, the shaft proximal end coupled to the hub distal end, wherein at least a portion of the shaft comprises a balloon inflation lumen and a first portion of a guidewire lumen;
a balloon having a balloon proximal end and a balloon distal end, the balloon proximal end coupled to the shaft distal end, wherein a second portion of the guidewire lumen extends at least partially through the balloon; and
a tip having a tip proximal end and a tip distal end, the tip comprising a medical-grade polymer, wherein:
a third portion of the guidewire lumen extends through the tip from the tip proximal end to the tip distal end,
the balloon distal end is bonded to the tip at a balloon-tip junction towards the tip proximal end, and
a tip length from the tip distal end to the balloon distal end is seven (7) to fifty (50) millimeters in a straightened state of the tip.

2. The balloon catheter of claim 1, wherein the tip length is twelve (12) to forty-five (45) millimeters in a straightened state of the tip.

3. The balloon catheter of claim 1, wherein the tip length is twenty-five (25) to thirty-five (35) millimeters in a straightened state of the tip.

4. The balloon catheter of claim 1, wherein the tip comprises a curvature in a relaxed state.

5. The balloon catheter of claim 4, wherein the curvature is ninety (90) to one hundred and eighty (180) degrees in the relaxed state.

6. The balloon catheter of claim 1, wherein:
the tip comprises a first durometer at the tip distal end and a second durometer at the tip proximal end, and
the first durometer is less than the second durometer.

7. The balloon catheter of claim 1, wherein the medical-grade polymer comprises:
polyurethane,
polyether block amide, and/or
low-density polyethylene (LDPE).

8. The balloon catheter of claim 1, wherein:
the tip comprises:
a first outer diameter at the tip distal end, and
a second outer diameter at the balloon-tip junction, and
the first outer diameter is smaller than the second outer diameter.

9. The balloon catheter of claim 1, wherein an outer diameter of the tip transitions from a first outer diameter at the tip distal end to a second outer diameter, greater than the first outer diameter, at the balloon-tip junction.

10. The balloon catheter of claim 1, wherein the tip comprises a truncated conical shape with a smallest outer diameter at the tip distal end.

11. The balloon catheter of claim 1, wherein:
the tip comprises a plurality of segments each having an outer diameter,
a first segment at the tip distal end has a first diameter,
a second segment at the ballon-tip junction has a second diameter,
a third segment between the first segment and the second segment has a third diameter,
the second diameter is greater than the third diameter, and
the third diameter is greater than the first diameter.

12. The balloon catheter of claim 11, wherein each of the plurality of segments is at least two (2) millimeters long.

13. The balloon catheter of claim 1, wherein:
the tip comprises a plurality of segments each having a durometer,
a first segment at the tip distal end has a first durometer,
a second segment at the ballon-tip junction has a second durometer,
a third segment between the first segment and the second segment has a third durometer,
the second durometer is greater than the third durometer, and
the third durometer is greater than the first durometer.

14. The balloon catheter of claim 13, wherein each of the plurality of segments is at least two (2) millimeters long.

15. The balloon catheter of claim 13, wherein:
the first durometer is between 70A and 40D,
the second durometer is between 45D and 55D, and
the third durometer is between 40D and 45D.

16. The balloon catheter of claim 1, wherein at least a portion of the outer surface of the tip is coated with a slip agent.

17. The balloon catheter of claim 16, wherein the slip agent comprises a hydrophilic coating.

18. The balloon catheter of claim 1, wherein at least part of the third portion of the guidewire lumen comprises a polytetrafluoroethylene (PTFE) liner.

19. The balloon catheter of claim 1, wherein at least part of the third portion of the guidewire lumen comprises a polyimide thin-walled tube.

20. The balloon catheter of claim 1, wherein the tip comprises a plurality of radiopaque markers.
